(19) United States
(12) Patent Application Publication
(10) Pub. No.: US 2006/0217813 A1
(43) Pub. Date: Sep. 28, 2006

(54) Facial implant

(76) Inventors: Jeffrey C. Posnick, Potomac, MD (US); Alma L. Coats, Delray Beach, FL (US)

Correspondence Address: STEPTOE & JOHNSON LLP, 1330 CONNECTICUT AVENUE, N.W., WASHINGTON, DC, 20036, US

(21) Appl. No.: 11/385,688

(22) Filed: Mar. 22, 2006

Related U.S. Application Data
(60) Provisional application No. 60/729,728, filed on Oct. 25, 2005.
(60) Provisional application No. 60/663,727, filed on Mar. 22, 2005.

(51) Int. Cl. A61F 2/28; B29C 67/04

(52) U.S. Cl. 623/17.18; 623/23.58; 264/119

(57) ABSTRACT

A facial implant includes fused polypropylene pellets. The pellets can be molded to a contoured shape. The shape can be used to augment, replace, or repair cranio-maxillofacial areas, such as the malar, mandibular angle, paranasal, nasal, temporal, cranial vault, orbital, ocular globe, and chin areas of a mammal, such as a human.

### CLAIM OF PRIORITY

This application claims priority under 35 USC § 119(e) to U.S. Patent Application Ser. No. 60/663,727 filed on Mar. 22, 2005 and U.S. Patent Application Ser. No. 60/729,728 filed on Oct. 25, 2005, each of which is hereby incorporated by reference.

### TECHNICAL FIELD

The present invention relates to polymeric materials for facial implants.

### BACKGROUND

Cosmetic and reconstructive facial implants are frequently manufactured from linear high-density polyethylene. The polyethylene implants can be porous to allow for tissue ingrowth. The implant shapes can be manufactured in a variety of shapes and volumes to augment or restore the contour of the cranio-maxillofacial skeleton, and to replace deficient soft tissue volume (e.g. ocular globe). While polyethylene has proven to be a versatile and useful plastic, its properties are not ideal for all applications. For this reason, chemists have investigated the polymerization of other olefin monomers, particularly monomers that possess a substituent group other than hydrogen on one of the olefinic carbon atoms. The polymers that result from these reactions possess different physical properties from those of polyethylene and have found important applications. Polypropylene has a higher melting point (150-173° C.), higher tensile strength, and greater rigidity than polyethylene. It is also less permeable than polyethylene to liquids and gases.

### SUMMARY

Materials to be used for a facial implant can include porous polypropylene. Materials of the facial implant include fused polypropylene pellets having a spherical shape. The facial implant can also have a contoured shape and a specific aspect ratio.

In one aspect, a facial implant includes fused polypropylene pellets having a spherical shape. In another aspect, a facial implant includes a volume having a contoured shape and an aspect ratio from about 1:3 to 1:20. In another aspect, a facial implant includes porous polypropylene. In another aspect, a method of manufacturing a facial implant includes molding pellets of polypropylene and fusing the pellets. In yet another aspect, a method of placing a facial implant into a mammal includes selecting a polypropylene implant comprising spherical pellets and placing the implant into a cranio-maxillofacial area.

The facial implant can include pellets that are molded to a volume having a contoured shape. The facial implant can have a uniform height, a varying height, or a maximum height that tapers toward at least one edge of the volume.

The facial implant can include a non-porous surface or a porous surface. The facial implant can also include an additive or a coating. The facial implant can also include a metal mesh, such as a titanium mesh.

The facial implant can be a customized surgical implant, a chin implant, a cranial vault implant, an ear implant, a temporal implant, a mandibular angle implant, a paranasal implant, a nasal implant, a malar implant, an orbital implant, or an ocular globe implant. The facial implant can include sintered polypropylene or porous polypropylene, which can be molded and further shaped or trimmed.

In one aspect, a facial implant can include an initial molded shape and a second shape. In another aspect, the initial shape can be determined from a mold. The second shape can be a customized shape. The customized shape can be a burred shape. A burred shape refers to a shape having a sculpted or shaven projected edge. The shape can be determined by or modified according to the implant's location or additional materials or grafts.

In another aspect, a method of manufacturing a facial implant includes molding pellets of polypropylene and fusing the pellets. In yet another aspect, a method of placing a facial implant into a mammal includes selecting a polypropylene implant comprising spherical pellets and placing the implant into a cranio-maxillofacial area.

The facial implant can include pellets that are molded to a volume having a contoured shape. The facial implant can have a uniform height, a varying height, or a maximum height that tapers toward at least one edge of the volume. The facial implant can include a non-porous surface or a porous surface. The facial implant can also include an additive or a coating. The facial implant can also include a metal mesh, such as a titanium mesh.

In another aspect, a method of manufacturing a polypropylene implant includes obtaining a polypropylene material, heating the material to a softening temperature, fusing the pellets to a molded shape, and manipulating the molded shape to a desired shape or a customized shape.

In certain circumstances, heating the material can include heating the material to a softening temperature. Fusing the pellets can include sintering. The pellets can include polypropylene flakes. The pellets can also include substantially monodisperse polypropylene particles.

In other circumstances, polypropylene can have a melt index that is sufficient to allow softening and fusing of the polypropylene pellets to provide a specific pore size. The pore size can be greater than 10 microns and less than 200 microns. The pore size can be greater than 50 microns and less than 150 microns.

Facial implants have often been made out of polyethylene (See e.g. U.S. Pat. No. 6,551,608, which is incorporated by reference herein). Polyethylene has been used a sintered porous material that may be molded to for different uses. (See e.g. U.S. Pat. No. 6,399,188, which is incorporated by reference herein). The disadvantage of polyethylene it that it has a lower softening temperature and it does not have stiffness of bone material. For example, it can soften at 82° C., which limits that amount of manipulation that can be performed on the product to create a customized shape. Ideally, an implant can be manipulated in any way to any customized shape. Facial implants in particular require a significant degree of customization because the facial features, surgical needs, and cosmetic preferences of every patient are unique. The ways in which an implant can be customized or manipulated can be limited by the physical and chemical properties of the implant material. One method known as burring refers to forming a projecting edge by shaving the implant to a sculpted form. Burring generates heat that can distort the shape of polyethylene, which has a lower softening temperature.

The advantage of polypropylene is that it can maintain its shape in temperatures as high as 104° C. Furthermore, a molded polypropylene implant can have stiffness more similar to that of bone material. The implant can have a bone-like feel, which makes it more durable and versatile for bone replacement or augmentation.

### DESCRIPTION OF DRAWINGS

FIG. 1 is a diagram depicting a cross section of a facial implant.

FIG. 2 is a diagram depicting a facial implant with tapered edges.

FIG. 3 is a diagram depicting a method of molding polymeric pellets to form a facial implant.

### DETAILED DESCRIPTION

A facial implant can be composed of porous polypropylene. The facial implant can include fused polypropylene pellets. The facial implant can have a specific aspect ratio and a contoured shape. The polypropylene pellets can have a spherical shape and can be molded to create facial implants of various shapes and volumes to augment, replace, or repair specific areas of a cranio-maxillofacial skeleton and facial soft tissues. Examples of cranio-maxillofacial areas include, but are not limited to, the chin, cranial vault, nasal, ear, orbital, paranasal, eyeball, angle of mandible, and malar (cheek/zygomatic) areas in a mammal, such as a human, for example.

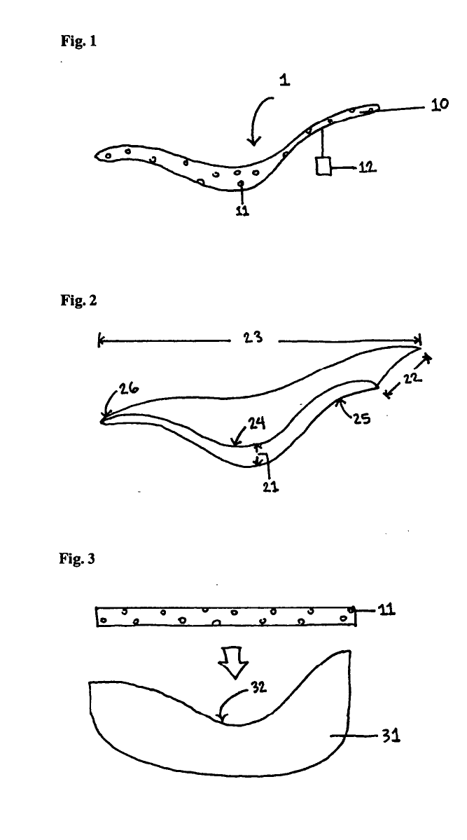

Referring to FIG. 1, a facial implant 1 can be made of a polymeric material 10 that is easily molded or shaped, resulting in a durable, porous and flexible material. In one embodiment, the facial implant includes polypropylene pellets 11, which are then molded or fused into an implant of a desired shape and volume. The facial implant can also include at least one functional additive 12 that can confer additional properties, such as strength, flexibility, and biocompatibility, to enhance the implant's performance. The pellets can be shaped into a facial implant by molding and fusing the pellets. In one embodiment, the pellets can be fused by sintering, for example.

Sintering is the process of bonding of adjacent surfaces of particles, such as pellets, by heating or applying pressure. Sintering can occur with softening, without melting, with melting, or with partial melting. Pellets of the same or of different polymers can be sintered together from a particular concentration, such that the particles join to each other at points of contact to form a coherent, porous mass, which, after being subjected to heat or pressure, can be easily shaped. Sinterable materials are described, for example, in U.S. Pat. No. 6,605,648 to Johnson, et al., which is incorporated by reference herein.

Referring to FIG. 2, a facial implant can have a total volume derived from a length 23, width 22, and height 21. A facial implant can have an aspect ratio ranging from 1:3 to 1:20. For example, if the height of the implant is 2 mm, then the width of the implant can range from 6 to 40 mm. The range can allow a person, such as a surgeon, to select an implant that has the necessary durability and flexibility to augment, replace, or repair a specific cranio-maxillofacial area of a mammal.

FIG. 2 also shows that the facial implant can be designed to have a contoured shape to complement a selected cranio-maxillofacial area of a mammal. The facial implant can be designed to have a subtle “S”-shape, which renders it suitable, for example, for augmenting a malar (cheek/zygomatic) bone of a mammal. The facial implant can have a main arc 24 and at least one minor arc 25, to create a shape that follows the contour of a specific cranio-maxillofacial area. The implant can have at least one tapered edge 26. In one embodiment, the facial implant can be positioned over the zygomatic arch and adjacent to the infraorbital nerve of a mammal. The subtle “S”-shape may be designed to augment either the right or left side of the cranio-maxillofacial skeleton. An implant can be composed of two units which attach together once in place at a desired location, such as a chin region, for example.

Referring to FIG. 3, a facial implant can be formed by molding polypropylene pellets 11 to a contoured shape 32 of a mold 31. The pellets can be fused by heat, or pressure, or a combination of both.

Polypropylene has several properties that make it advantageous as a facial implant: it is thermoplastic, biocompatible, durable, inexpensive, easily shaped, and resists deformation. Polypropylene has a higher melting point (150-173° C.), higher softening point (110-170° C.), higher polymer melt index (2.0-50.0), higher tensile strength, and greater rigidity than polyethylene. It can also be less permeable than polyethylene to liquids and gases. Because of the aforementioned characteristics, polypropylene can be used as a biomaterial while requiring fewer additives compared to polyethylene.

Polypropylene can be particularly advantageous as a facial implant because it has a lower density, ranging from approximately 0.880 to 0.920 grams per cubic inch, in comparison to other thermoplastic materials and high density polyethylene (HDPE), thus allowing for potential weight reductions. Polypropylene can have a high heat resistance and can be used in continuous environments as high as 220° F. (104° C.).

Polypropylene can also be highly resistant to chemical attack from solvents and chemicals in very harsh environments. In general, polypropylene is not susceptible to environmental stress cracking, and it can be exposed under load in the toughest environments. Resistance to weathering may be limited without the use of ultraviolet light absorbers, or stabilizers.

Polypropylene does not need drying prior to molding as opposed to most thermoplastic materials because polypropylenes are not hygroscopic. Therefore, a processor can work with the polypropylene material out of the container rather than having to add an initial step for drying the material. Furthermore, the excellent fatigue resistance and flexural modulus of polypropylene can make it a particularly suitable material for a surgical implant.

There are two primary types of polypropylene: homopolymer and copolymer. Homopolymer polypropylene can have a higher tensile strength than copolymer polypropylene and it is less costly. Copolymer polypropylene can have a higher impact strength but a lower tensile strength.

Unlike polyethylene, polypropylene will not polymerize via by free radical polymerization. Polypropylene can be made from the monomer propylene by Ziegler-Natta polymerization and by metallocene catalysis polymerization, or other methods, which are known in the art. Propylene can be fed to a nitrogen-blanketed reactor. The typical Ziegler-Natta catalysts, which can include TiCl₃ or TiCl₄, are used in a hydrocarbon media. Hydrocarbon solvents can be fed to the reactor. The typical temperature range of the reactor is 370° to 430° F. The reactor pressure can range from 250 to 350 psi, depending on the utilized commoners and solvents. The manufacturing process can be used as a continuous or a semicontinuous operation. After the reaction, the unreacted propylene and solvent can be removed, typically under a vacuum to ensure complete removal. Solvents can be sent to the solvent recovery system. The reaction product is chilled with water and passed through the cutter system. Companies can use different technologies to shape/pelletize the final products. Depending on the catalyst and the polymerization method used, the molecular configuration can be altered to produce various types of polypropylene, such as atactic, isotactic, syndiotactic, and elastomeric polypropylene.

Atactic polypropylene is characterized as a tacky polymer with amorphous behavior and low molecular weight. With atactic polypropylene, the pendent methyl groups are arranged randomly along the backbone of the molecule. Atactic polypropylene can be incorporated in adhesive, sealant, asphalt modification and roofing applications. Atactic polypropylene can also provide the same effect as a plasticizer, by reducing the crystallinity of the polypropylene. A small amount of atactic polymers in the final polymer can be used to improve certain mechanical properties. This can provide beneficial properties to the final polymer, such as improved low temperature performance, elongation, processability and optical properties.

Syndiotactic polypropylene can be produced in the laboratory and is manufactured, for example, by Arkema Canada, Inc. It has not been commercially used to the same degree as other forms of polypropylene.

Isotactic polypropylene has stereoregular configuration of the pendent methyl groups, and this configuration provides crystallinity in the polymer. Many of polypropylene's mechanical properties and processability can be determined by the level of isotacticity. The increased crystallinity of polypropylene can provide a higher flexural modulus, and tensile properties much higher than polyethylene.

Elastomeric homopolypropylene has a combination molecular structure of isotactic and atactic polypropylene. This configuration can provide elasticity in the polymer and a combination of isotactic and atactic polypropylene properties.

The basic difference between polypropylene and other thermoplastic materials such as polycarbonate, polycarbonate/ABS blends and polystyrene, is that polypropylene is a semicrystalline polymer, whereas other thermoplastic materials are classified as amorphous polymers.

Due to its higher crystallinity, polypropylene has excellent moisture barrier properties and good optical properties. High crystallinity imparts improved chemical resistance in comparison to amorphous polymers. Therefore, polypropylenes can be exposed to a wide variety of agents without failure in comparison to amorphous polymers. Part shrinkage for polypropylene is higher than for amorphous polymers. This is due to better packing of the molecular chains in the crystalline regions. Differences in cooling lead to differences in crystallinity and thus differences in shrinkage. Therefore, controlling process variables, such as mold temperature and cooling time, plays a major role in determining mold shrinkage for semi-crystalline materials such as polypropylene.

Polypropylene can crystallize by forming branched structures which grow until they either exhaust the supply of crystallizing material or affect their surroundings such as to prevent further crystallization from occurring. The crystals grow by branching the degree of which depends upon temperature, chain branch structure, concentration, and nature of surrounding material (solvent or melt). At low concentration, these may interlock, forming a space-filling structure. Any remaining crystallizable polymer can then fill in the spaces between crystals within this network. Noncrystallizable material can remain within this structure. If this material is a volatile solvent, its evaporation can lead to a foam.

Polypropylene may be linear or branched. Linear polypropylene can have a relatively low level of melt strength and melt drawability. Branched propylene polymer can have a very high melt strength in combination and a relatively higher melt extensibility. With blends of linear and pure branched propylene polymers, the melt strength, melt extensibility and strain hardening behavior can increase with the amount of branched polypropylene.

The growth and morphology of polypropylene structures can be followed by the combined use of wide-angle x-ray diffraction, small-angle x-ray scattering, and small-angle light scattering. For following kinetics, synchrotron x-ray sources can be employed. In the case of foams, surface areas can be measured using gas adsorption techniques.

Polypropylene can be blended with other additives, other polymeric materials or functional additives. Other additives and polymeric materials that may be blended with polypropylene are disclosed in U.S. Pat. No. 5,929,129, which is incorporated by reference herein. Other polymeric materials can include, for example, low density polyethylene, high density polyethylene, linear low density polyethylene, medium density polyethylene, polypropylene, ethylene propylene rubber, ethylene propylene diene monomer terpolymer, polystyrene, polyvinyl chloride, polyamides, polyacrylics, cellulosics, polyesters, and polyhalocarbons. Copolymers of ethylene with propylene, isobutene, butene, hexene, octene, vinyl acetate, vinyl chloride, vinyl propionate, vinyl isobutyrate, vinyl alcohol, allyl alcohol, allyl acetate, allyl acetone, allyl benzene, allyl ether, ethyl acrylate, methyl acrylate, methyl methacrylate, acrylic acid, and methacrylic acid may also be used. Various polymers and resins which find wide application in peroxide-cured or vulcanized rubber articles may also be added, such as polychloroprene, polybutadiene, polyisoprene, poly(isobutylene), nitrile-butadiene rubber, styrene-butadiene rubber, chlorinated polyethylene, chlorosulfonated polyethylene, epichlorohydrin rubber, polyacrylates, and butyl or halo-butyl rubbers. Other resins are also possible, as will be apparent to one skilled in the art, including blends of the above materials. Any or all of the additional polymers or resins may be advantageously grafted or cross-linked, in concert or separately, within the scope of the object of this invention.

The Composition Distribution Breadth Index (CDBI) is a measurement of the uniformity of distribution of comonomer to the copolymer molecules, and is determined by the technique of Temperature Rising Elution Fractionation (TREF), as described in, for example, Wild et. al., J. Poly. Sci., Poly. Phys. Phys. Ed., Vol. 20, p. 441 (1982). This attribute relates to polymer crystallizability, optical properties, toughness and many other important performance characteristics of compositions of the present art. For example, a polyolefin resin of high density with a high CDBI would crystallize less readily than another with a lower CDBI but equal comonomer content and other characteristics, enhancing toughness in objects of the polymeric material.

Polypropylene is biocompatible and has been used successfully in the human body as a mesh for hernia repair, such as the DAVOL BARD® Mesh, which is commercially available. Medical literature, such as Law NW, Ellis H., A comparison of polypropylene mesh and expanded polytetrafluoroethylene patch for the repair of contaminated abdominal wall defects—An experimental study. Surgery 1991; 109:652-5, also shows polypropylene combined with polytetrafluoroethylene being used to repair the abdominal wall.

A facial implant can be formed by obtaining polypropylene pellets, molding the pellets to a contoured shape, and fusing the pellets. A compression mold can be used to fuse the pellets, for example, by sintering. Sintering is the process of bonding of adjacent surfaces of particles, such as pellets, by heating or applying pressure. Sintering may occur with softening, without melting, with melting, or with partial melting. Polypropylene can be particularly suitable for sintering because of its relatively low melting temperature and their low thermal conductivity.

Polypropylene pellets can be obtained and heated to a softening temperature. A softening temperature for polypropylene can be greater than 82° C., greater than 110° C., and greater than 140° C. The polypropylene pellets can include substantially monodisperse polypropylene particles. Fusing the substantially monodisperse polypropylene particles can yield a molded implant with substantially uniform pore sizes. The pore size upon fusing or sintering depends on the particle size used. Generally, smaller particles sizes can yield smaller pores. Furthermore, a uniform particle size can yield a uniform porosity, thereby ensuring that voids in the implant are not filled due to a difference in particle sizes.

Pellets of the same or of different polymers can also be sintered together from a particular concentration, such that the particles join to each other at points of contact to form a coherent, porous mass, which, after being subjected to heat or pressure, can be easily shaped. The mold can be heated to the sintering temperature of the selected polymer. Polypropylene pellets can be heated to the softening temperature, heated to the sintering temperature, or subjected to a pressure, or a combination of heat and pressure. The various processes, or combinations thereof can cause a degree of softening, which results in the material conforming to the contour of the mold.

The resulting polypropylene product can have an initial shape and a customized shape. Polypropylene has a higher softening temperature (generally 110-170° C.) and is generally stiffer than polyethylene, which renders it more durable, versatile and suitable for further manipulation. The disadvantage of polyethylene it that it has a lower softening temperature. For example, it can soften at 82° C., which limits that amount of manipulation that can be performed on the product to create a customized shape. Ideally, an implant can be manipulated in any way to any customized shape. Facial implants in particular require a significant degree of customization because the facial features, surgical needs, and cosmetic preferences of every patient are unique. One method known as burring refers to forming a projecting edge by shaving the implant to a sculpted form. A burred shape refers to a sculpted form. Burring generates heat that can distort the shape of polyethylene, which has a lower softening temperature.

The advantage of polypropylene is that it can maintain its shape in temperatures as high as 100° C. Polypropylene, which has a density of approximately 0.9 g/mL, can be used to form a molded polypropylene product can have stiffness similar to that of bone material. The product can have a bone-like feel, which gives it the durability and versatility for shaping. The initial shape of the product can follow that of the mold. The initial shape can be manipulated to a customized shape based on a desired shape, implant location, or additional materials or grafts to be used.

The polymer can then be allowed to equilibrate, and can subsequently subjected to additional pressure, depending on the desired pore size. Typically, a greater pressure and a higher temperature, for longer time periods can result in a smaller pore size and greater mechanical strength. Once a porous material has been formed, the mold can be allowed to cool. If the mold was subjected to pressure, the cooling can occur while it is being applied or after it has been removed. The material can be removed and then optionally processed. Examples of processing can include, sterilizing, shaping, cutting, trimming, polishing, milling, encapsulating, and coating.

Polypropylene pellets can be selected based on their specific melt index. For example, a melt index can be high enough such that material can be heated, softened, fused, or sintered to provide a specific pore size. The pore size can be greater than 10 microns and less than 250 microns. The pore size can be greater than 50 microns and less than 150 microns.

A melt index refers to the number of grams of a thermoplastic resin which can be forced through a 0.0825 inch orifice when subjected to 2160 grams force in 10 minutes at 190° C. Generally, a higher molecular weight will yield a lower melt index. For example, polypropylene with a molecular weight of 580,000 can have a melt index of 0.5, while a molecular weight of 174,000 can have a melt index of 2.2, and a molecular weight of 127,000 can have a melt index of 4.5. Polypropylene for use in a facial implant can have a melt index high enough to allow that material to be heated, softened, fused, or sintered to a specified pore size. Generally, a higher melt index will result in a smaller pore size. For example, if a pore size between 50 and 150 microns is desired, an appropriate melt index can extrapolated from existing data, or determined experimentally, choosing polypropylene materials with increasing melt indexes until a desire pore size is reached. A polypropylene material can be selected based on its melt index, based on any desired pore size. For example, the melt index can be greater than 0.5 and less than 50. Pore size refers to the size of the holes or voids between powder particles or pellets, often measured by mercury porosimetry on open pores.

Besides the size of the original particles, the porosity of the sintered material can also be controlled by using blends of high and low melt flow materials. In some cases, high melt polymers can determine the average pore size, while the low melt polymer can give the material enhanced structural strength. Besides using blends of similar and dissimilar polymers with high and low melt flow rate, it is also possible to add other types of particulate materials in the matrix that impart other properties to the implant structure

A softening point, or the Vicat Softening Point, refers to the temperature at which a flat-ended needle of 1 square millimeter circular or square cross section will penetrate a thermoplastic specimen to a depth of 1 mm under a specified load using a uniform rate of temperature rise. (ASTM D-1525-58T).

The pellets can be molded to form a single layer or more than one layer. By coating the first layer with additional pellets and likewise subjecting the second layer to heat or pressure, a second layer can be molded and bonded to the first layer.

Suitable molds are commercially available. Suitable mold materials include, but are not limited to, metal alloys such as aluminum and stainless steel, high temperature materials, and other materials known in the art. Specific molds can have varying heights and diameters.

A facial implant can be made of porous polypropylene. The pores can range in size depending on, for example, the degree of flexibility or strength desired. The pore sizes can be controlled, for example by a selected temperature, pressure, exposure time, or a combination of the above. The facial implant can have similarly sized particles or blends of particle sizes of polypropylene, as is described in U.S. Pat. No. 6,083,618, which is incorporated by reference herein. The particle size distribution can be determined by commercially available screens. The particles can include polypropylene alone or polypropylene blended with other materials, other polymers, or other functional additives. The particles can be monodisperse, which can result in pore sizes having a nearly identical size and a narrow size distribution. The particles can also be polydisperse, which can result in pore sizes with a wider size distribution.

There are several methods for making porous substrates, such as sintering, using blowing agents, reverse phase precipitation, and microcell formation, such as those described by U.S. Pat. Nos. 4,473,665 and 5,160,674, which are incorporated by reference herein.

A facial implant can be formed from particles or pellets that are monodisperse, having a narrow size distribution. The facial implant can also be made from particles or pellets that are polydisperse, having a wider size distribution. In one embodiment, the size distribution of the composite material particles can also be about one order of magnitude or more (expressed in micrometers). Thus, for example, if the average particle size of the composite material particles is about 20 micrometers, the composite particles can range in size from, for example, about 0.1 to about 50 micrometers. This can promote good packing of the particles and can contribute to a particularly preferred fast-hardening effect.

A facial implant can be porous or nonporous. A facial implant can have a substantially uniform porosity. Pore sizes can range, for example, between 100 to 1000 micrometers. Uniform porosity can be beneficial in a facial implant because tissue ingrowth is more likely to progress evenly throughout materials with a uniform porosity, rather than materials that contain distinct areas of high and low permeability. A substantially uniform porosity can also achieve a more uniform mechanical strength, by avoiding regions in the implant that would be disproportionately vulnerable to stress. The average pore size and density can be determined, for example, using a mercury porosimeter or scanning electron microscopy.

A facial implant can include pellets that have been made by underwater pelletizing. Underwater pelletizing is described, for example, in U.S. patent application Ser. No. 09/064,786, filed Apr. 23, 1998, and U.S. Provisional Patent Application No. 60/044,238, filed Apr. 24, 1999, both of which are incorporated by reference herein This method can be used to produce particles with diameters of about 36 micrometers, and it offers several advantages. First, underwater pelletizing provides accurate control over the average size of the particles produced, in many cases thereby eliminating the need for an additional screening step and reducing the amount of wasted material. A second advantage of underwater pelletizing is that it allows significant control over the particles' shape.

Thermoplastic particle formation using underwater pelletizing typically requires an extruder or melt pump, an underwater pelletizer, and a drier. The thermoplastic resin is fed into an extruder or a melt pump and heated until semi-molten. The semi-molten material is then forced through a die. As the material emerges from the die, at least one rotating blade cuts it into pieces herein referred to as “pre-particles.” The rate of extrusion and the speed of the rotating blade(s) determine the shape of the particles formed from the pre-particles, while the diameter of the die holes determine their average size. Water, or some other liquid or gas capable of increasing the rate at which the pre-particles cool, flows over the cutting blade(s) and through the cutting chamber. This coagulates the cut material (i.e., the pre-particles) into particles, which are then separated from the coolant (e.g., water), dried, and expelled into a holding container.

The average size of particles produced by underwater pelletizing can be accurately controlled and can range from about 0.014″ (35.6 micrometers) to about 0.125″ (318 micrometers) in diameter, depending upon the porous substrate. Average particle size can be adjusted simply by changing dies, with larger pore dies yielding proportionally larger particles. The average shape of the particles can be optimized by manipulating the extrusion rate and the temperature of the water used in the process.

While the characteristics of a porous material can depend on the average size and size distribution of the particles used to make it, they can also be affected by the particles' average shape. Consequently, in one embodiment, the particles of plastic and functional additive particles are substantially spherical. This shape facilitates the efficient packing of the particles within a mold. Substantially spherical particles, and in particular those with smooth edges, also tend to sinter evenly over a well-defined temperature range to provide a final product with desirable mechanical properties and porosity.

The polymer pellets can be substantially spherical and free of rough edges. In another embodiment, the polymer pellets and functional additives combined, are substantially spherical and free of rough edges. The pellets can be thermal fined to ensure smooth edges, and can be screened to ensure a proper average size and size distribution. Thermal fining is a well-known process wherein particles are rapidly mixed and optionally heated such that their rough edges become smooth. Mixers suitable for thermal fining include the W series high-intensity mixers available from Littleford Day, Inc., Florence, Ky.

Particles made by underwater pelletizing, which allows precise control over particle size and can yield smooth, substantially spherical particles, typically do not need to be thermal fined or screened.

A facial implant can be selected for its desired biocompatibility, strength, flexibility, and resistance to degradation. The facial implant can also avoid undesirable reactions such as, but not limited to, thrombus formation, tumor formation, allergic reactions, and inflammation. A facial implant can maintain its physical properties during the time that is remains implanted in the cranio-maxillofacial tissues.

A facial implant can include a non-porous surface or a porous surface. A facial implant can include an additive or a coating.

In one embodiment, a facial implant can include a functional additive. Functional additives are materials that contain functional groups such as, but not limited to, hydroxyl, carboxylic acid, anhydride, acyl halide, alkyl halide, aldehyde, alkene, amide, amine, guanidine, malemide, thiol, sulfonate, sulfonic acid, sulfonyl ester, carbodiimide, ester, cyano, epoxide, proline, disulfide, imidazole, imide, imine, isocyanate, isothiocyanate, nitro, or azide. Functional additives can confer additional properties to enhance the polymer's performance as a biomaterial.

Preferred functional additives can be incorporated into the porous material without degrading or losing functionality when subjected to heat or pressure or once implanted.

A facial implant can include a synthetic coating or a biological coating. The coating can enhance the performance of the facial implant, for example, by increasing mechanical strength, promoting cell growth, promoting biomolecule immobilization, increasing resistance to infection, improving lubricity, improving anti-thrombogenicity, and promoting stem cell or osteoblast differentiation. The coating can also allow precise biomolecular interactions to be initiated or modulated. Such coatings are commercially available, for example, from AFFINERGY™.

The coating can be applied on any surface of the facial implant, such as an external surface or between polymeric layers within the implant. Biological coatings can include, for example, cell receptors, growth factors, chondrocytes, proteins, enzymes, and antibodies. Synthetic coatings can include for example, titanium, stainless steel, TEFLON®, LATEX®, collagen, PET, PETG, PGA, polystyrene, polycarbonate, glass, or nylon.

The coating can also include an interfacial biomaterial that contains modular surfaces, such as two functional peptides that can bind to a bioactive material and to a synthetic material. The two peptides can be joined by a linker that can provide cross-linking or cleaving capabilities. Interfacial biomaterials are commercially available, for example, from AFFINERGY™.

In one example, a facial implant can include a coating, which is designed to specifically recruit and anchor osteoblasts to the implant surface. The coating can further induce differentiation of osteoblasts into bone cells using immobilized growth factors, such as BMP-2 or BMP-7. The coating can further immobilize stem cells and promote stem cell differentiation into a desired cell type, such as mineralized bone. The coating can also minimize or prevent the attachment of undesirable cells and bacteria to the implant.

A facial implant can include a metal mesh, such as a titanium mesh. The mesh can be positioned on any surface of the facial implant or in between polymeric layers of the implant.

The relative amounts of polymer and additive used can vary with the specific materials used, the desired strength and flexibility of the implant, and the properties conferred by a selected additive.

In one embodiment, the polymer and functional additive are admixed then fused. In another embodiment, other materials may also be mixed into the polymer and functional additive before sintering. Depending on the desired sized and shape of the final product, this can be accomplished using a mold or a belt line disclosed by U.S. Pat. No. 3,405,206, which is incorporated by reference herein.

A facial implant can be customized. For example, a customized implant can be designed based on 3-dimensional computed tomography (CT) scan models to make the implant patient-specific. CT or computer-aided design allows one to design a customized implant by obtaining information about the site of an implant (i.e. by scanning). Scanning can include using an MRI, an ultrasonic device, an x-ray machine, a camera, a scope, and combinations thereof to obtain information about the site of an implant. After information is obtained, one can process the information to generate information on the size and shape of the implant. After information is obtained and processed, one can transfer at least a portion of the generated information to a mold in order to form, at least partially, a custom implant from a moldable compound. A mold or a molding machine can include at least one mold cavity that can be varied in size or shape. The size or shape of the mold cavity can be adjusted or changed based at least partially on the data transferred to the mold or the molding machine, resulting in a customized shape. An example of using computer-aided design for prosthetic implants can be found in U.S. Pat. No. 6,786,930, which is hereby incorporated by reference.

The implant can also be customized by shaping, shaving, trimming, or burring the implant according to a desired shape. A burred shape refers to a sculpted or customized shape. The implant may also be modified according to additional materials or grafts that may be involved in a surgical procedure.

A facial implant can have a specific shape and aspect ratio, which renders it particularly suitable for implanting in cranio-maxillofacial areas. In one embodiment, a facial implant can be designed to have a subtle “S” shape, which renders it suitable, for example, for augmenting or repairing the malar bone. The implant can have a main arc and at least one minor arc to the implant to follow the contour of a cranio-maxillofacial area. The implant can have at least one tapered edge. In one embodiment, the implant can be positioned over the zygomatic arch and adjacent to the infraorbital nerve. The subtle “S” shape of the implant can be designed to augment either the right or left side of the cranio-maxillofacial skeleton. The facial implant can be a customized surgical implant, a chin implant, a cranial vault implant, an ear implant, a temporal implant, a mandibular angle implant, a paranasal implant, a nasal implant, a malar implant, an orbital implant, or an ocular globe implant. The facial implant can be contoured or anatomical. For example, the malar implant can be shaped to augment, replace, or repair, the cheek and zygomatic areas of the cranio-maxillofacial skeleton. The ocular globe implant can be round or conical. The chin implant can be contoured or extended. The mandibular angle implant can be contoured, and the nasal and paranasal implants can have a crescent shape.

The implant can be molded to various heights and volumes within the specified aspect ratio. A facial implant can have a total volume derived from a length, width, and height. A facial implant may be molded to have an aspect ratio ranging from 1:3 to 1:20. For example, if the maximum height or thickness of the implant is 2 mm, the width of the implant can range from 6 to 40 mm. This range can allow a surgeon to select an implant that has the necessary durability and flexibility to augment, contour, or replace a specific cranio-maxillofacial area.

A facial implant may be molded to have a uniform height, or a varying height. In one embodiment, a facial implant can have a varying height, where the maximum height tapers to at least one edge of the implant. In another embodiment, a facial implant can have a subtle “S” curve with a varying height, where the maximum height tapers to at least one edge of the implant.

An implant can also have a varying height and a substantially uniform porosity, thereby allowing even tissue ingrowth while effectively following the natural arch of the malar bone.

Other embodiments are within the scope of the following claims.

What is claimed is:

1. A facial implant comprising fused propylene pellets having a spherical shape.

2. The implant of claim 1, wherein the pellets are molded to a volume having a contoured shape.

3. The implant of claim 1, wherein the implant has a uniform height.

4. The implant of claim 2, wherein the implant has a maximum height that tapers toward at least one edge of the volume.

5. The implant of claim 1, wherein the implant is a customized surgical implant.

6. The implant of claim 1, wherein the implant is a chin implant.

7. The implant of claim 1, wherein the implant is a cranial vault implant.

8. The implant of claim 1, wherein the implant is an ear implant.

9. The implant of claim 1, wherein the implant is a temporal implant.

10. The implant of claim 1, wherein the implant is a mandibular angle implant.

11. The implant of claim 1, wherein the implant is a paranasal implant.

12. The implant of claim 1, wherein the implant is a nasal implant.

13. The implant of claim 1, wherein the implant is a malar implant.

14. The implant of claim 1, wherein the implant is an orbital implant.

15. The implant of claim 1, wherein the implant is an ocular globe implant.

16. The implant of claim 1, further including at least one nonporous or porous surface.

17. The implant of claim 1, further including a metal mesh.

18. The implant of claim 17, wherein the metal is titanium.

19. The implant of claim 1, further including an additive or a coating.

20. A facial implant comprising a volume having a contoured shape and an aspect ratio from about 1:3 to 1:20.

21. The implant of claim 20, wherein the volume includes fused polypropylene.

22. The implant of claim 20, wherein the volume includes porous polypropylene.

23. The implant of claim 20, wherein the volume is a customized surgical implant.

24. The implant of claim 20, wherein the volume has a uniform height.

25. The implant of claim 20, wherein the volume has a varying height.

26. The implant of claim 20, wherein the volume has a maximum height that tapers toward at least one edge of the volume.

27. The implant of claim 20, further including at least one nonporous or porous surface.

28. The implant of claim 20, further including a metal mesh.

29. The implant of claim 28, wherein the metal is titanium.

30. The implant of claim 20, further including an additive or a coating.

31. A facial implant comprising porous polypropylene.

32. The implant of claim 31, further including at least one nonporous or porous surface.

33. The implant of claim 31, further including a metal mesh.

34. The implant of claim 33, wherein the metal is titanium.

35. The implant of claim 31, further including an additive or a coating.

36. A method of manufacturing a facial implant comprising molding pellets of polypropylene and fusing the pellets.

37. A method of placing a facial implant into a mammal comprising: selecting a polypropylene cranio-maxillofacial implant comprising spherical pellets; and placing the implant into a cranio-maxillofacial area.

38. The method of claim 37, further comprising shaping or trimming the implant.

39. A facial implant comprising fused polypropylene pellets having an initial molded shape and a second shape.

40. The implant of claim 39, wherein the pellets include polypropylene flakes.

41. The implant of claim 39, wherein the pellets include substantially monodisperse polypropylene particles.

42. The implant of claim 39, wherein the initial molded shape is a contoured shape determined from a mold.

43. The implant of claim 39, wherein the second shape is a customized shape.

44. The implant of claim 43, wherein the customized shape is a burred shape.

45. The implant of claim 43, wherein the customized shape is determined by the implant location.

46. The implant of claim 43, wherein the customized shape is determined by additional materials or grafts.

47. A method of manufacturing a facial implant having fused polypropylene pellets comprising obtaining a polypropylene material, heating the material, fusing the pellets to a molded shape and manipulating the material to a desired shape.

48. The method of claim 47, wherein heating the material includes heating the material to a softening temperature.

49. The method of claim 47, wherein fusing the pellets includes sintering.

50. The method of claim 47, wherein the pellets include polypropylene flakes.

51. The method of claim 47, wherein the pellets include substantially monodisperse polypropylene particles.

52. The method of claim 47, wherein manipulating includes burring the implant.

53. The method of claim 52, wherein burring includes forming a projected edge on the implant.

54. The method of claim 47, wherein the polypropylene pellets have a melt index that is sufficient to allow softening and fusing of the pellets to provide a specific pore size.

55. The method of claim 54, wherein the pore size is greater than 10 microns and less than 200 microns.

56. The method of claim 54, wherein the pore size is greater than 50 microns and less than 150 microns.
